(19) United States
(12) Patent Application Publication
(10) Pub. No.: US 2018/0113427 A1
(43) Pub. Date: Apr. 26, 2018

(54) APPARATUS AND METHOD FOR SAFEGUARDING USERS DURING USE OF APPARATUS HAVING AT LEAST A PART IN MOTION

(71) Applicant: FUTURE SMART S.r.l., ZEVIO (Verona) (IT)

(72) Inventor: Maurizio MARCATO, Velo Veronese (IT)

(21) Appl. No.: 15/567,309

(22) PCT Filed: Apr. 12, 2016
(86) PCT No.: PCT/IB2016/052065

§ 371 Date: Oct. 17, 2017

(30) Foreign Application Priority Data
Apr. 20, 2015 (IT) VR2015A000063

(51) Int. Cl. G05B 19/048; G06F 1/16; G07C 9/00; A61B 5/024; A61B 5/00

(52) U.S. Cl. CPC G05B 19/048; G06F 1/163; G05B 2219/24024; A61B 5/02438; A61B 5/681; G07C 9/00158

(57) ABSTRACT

Described is a management apparatus for safeguarding users during use of apparatuses that include a part in motion. The management apparatus includes a management device that manages the part the motion during use of the management apparatus. The management device is configurable between an enabling condition of the motion and a condition of interrupting the motion. The management apparatus may also include a device for measuring at least one biometric value associated with the body of the user while using the management device and/or the apparatus that includes a part in motion. The management device may also include a processing unit operatively connected to the management device and to the measuring device. The processing unit is configured to interrupt the movement of the part in motion when the measured biometric value moves away from reference biometric value.

This invention relates to an apparatus and a method for safeguarding users during use of apparatus having at least a part in motion. More specifically, this invention belongs to the field of safety at work or at home in relation to the use of tools which involve use by the users. Examples of these electrical tools are: drills, electric saws, nail guns, . . . .

Preferably, this invention relates to the sector of electrical tools wherein the motion is powered by electricity. However, this invention may also be used for apparatuses wherein the moving part is moved by means of a combustion engine (for example a chain saw) or by other means not expressly mentioned herein.

Reference is made hereinafter mainly to tools of the electric type, without thereby excluding the possibility of tools powered by a combustion engine or other means.

In effect, according to the prior art, during use of electrical tools the users can injure themselves due to the movement of the tool itself. For example, if the tool is a drill, the user's hand can remain trapped at the drill bit of the drill due, for example, to wrapping of the glove, and then twisting on itself, causing fracturing of the hand, wounds, amputations, etc.

More specifically, during the use of a tool, the user can be injured due to the fact that the movement of the tool continues even when the user has already found him/herself in a dangerous condition. However, the user is so concentrated on the area for attaching/fixing to the part of the body that instead of disabling the tool (for example by releasing the pushbutton of the drill) the user tries to free the part of the body in danger (thereby increasing even further the risk of injury).

Some examples of devices for safeguarding users are described in patent document US 2012/0319846 and involve the use of a proximity sensor (to be applied to the user) to check the distance of the user from the apparatus in such a way as to switch the apparatus ON/OFF only if the user is in the correct position.

In this situation, the aim of this invention is to provide an apparatus and a method for safeguarding users during the use of apparatuses with at least a part in motion which overcomes the above-mentioned drawbacks.

More specifically, the aim of this invention is to provide an apparatus and a method for safeguarding users during the use of apparatuses with at least a part in motion which allows the physical damage caused by these apparatuses to be reduced in the case of danger.

Another aim of this invention is to provide an apparatus and a method for safeguarding users during the use of apparatuses with at least a part in motion which allows the management of the electrical apparatus to be optimised in the case of danger.

The aims indicated are substantially achieved by an apparatus and a method for safeguarding users during the use of apparatuses with at least a part in motion as described in the appended claims.

Further characteristic features and advantages of this invention will emerge more clearly from the detailed description of several preferred, but not exclusive embodiments of an apparatus and a method for safeguarding users during use of apparatus having at least a part in motion illustrated in the accompanying drawings, in which:

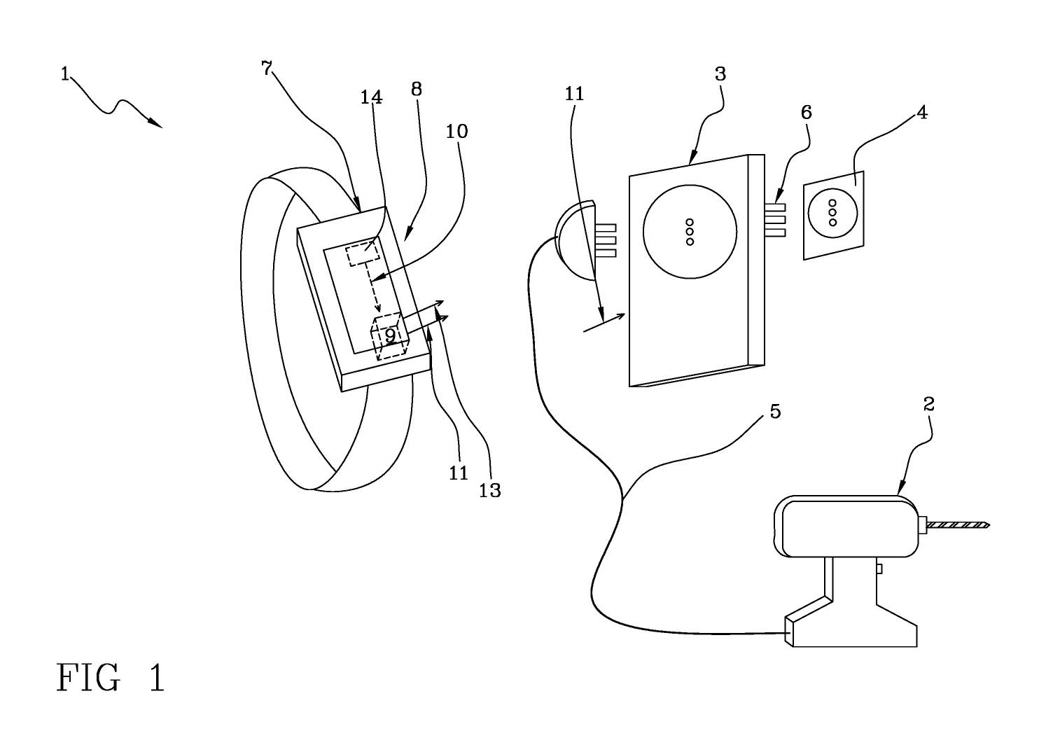

FIG. 1 is a schematic view of a first preferred embodiment of the apparatus according to this invention;

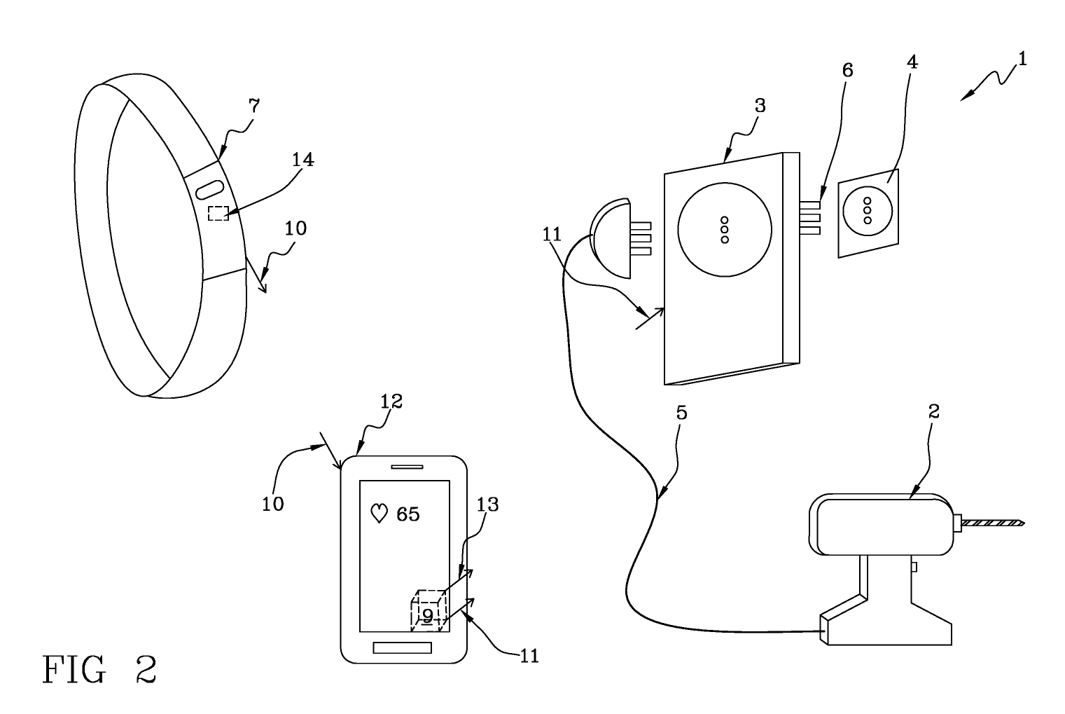

FIG. 2 is a schematic view of a second preferred embodiment of the apparatus according to this invention; and

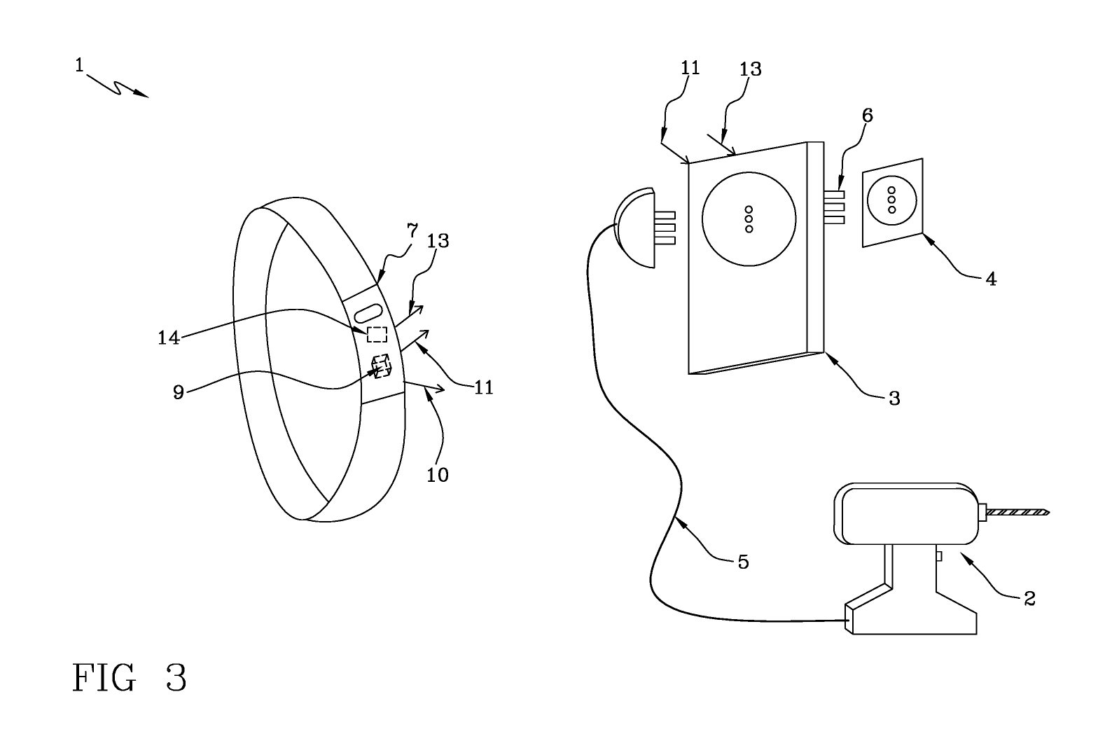

FIG. 3 is a schematic view of a third preferred embodiment of the apparatus according to this invention.

With reference to the above-mentioned drawings, the numeral 1 denotes in its entirety an apparatus 1 for safeguarding users during use of apparatuses 2 with at least a part in motion according to this invention.

More specifically, the apparatus 1 comprises a management device 3 of the part in motion in use connected to the part in motion of the apparatus 2 to the apparatus 2. More specifically, the management device 3 is configurable between an enabled condition during which the motion of the part of the apparatus 2 is enabled, and an interruption condition during which the motion of the part of the apparatus 2 is disabled. In detail, the management device 3 may be connected to a system for controlling the moving part of the apparatus 2 irrespective of the type of movement means connected to the part in motion. In other words, the movement means may be defined by an electric motor, an actuator, a switch, a combustion engine or other . . . .

For example, in the case of an apparatus 2 powered by a combustion engine (for example, a chain saw) the management device 3 is configured to act on the combustion engine (for example by means of an actuator) in order to manage the movement.

In any case, the management device 3 can be connected to a panic element (for example, a button) which is often connected to the industrial apparatuses 2 for stopping them if they are deactivated. In effect, in this case, the panic element already comprises a system for controlling the apparatus 2.

In any case, the management device 3 may act directly on an electrical control circuit of the apparatus 2 by means of electrical signals or may act mechanically on the part in motion by means of a suitable actuator.

In the case shown in the accompanying drawings, the management device 3 is interposed and connected between a source 4 of electric current and an electrical apparatus 2. The accompanying drawings show the source 4 of electricity (by way of example represented by an electrical socket) and an electrical apparatus 2 (drill).

In that case, the flow of electric current from the source 4 of electric current towards the apparatus 2 is enabled during the enabling condition, whilst there is no flow of electric current from the source 4 of electric current towards the apparatus 2 during the interruption condition.

It should be noted that the management device 3 is connected to the electric apparatus 2 by an electric cable 5. Thus, the management device 3 is interposed between the electricity socket and the electric cable 5 which leads to the electrical apparatus 2.

Preferably, the management device 3 comprises an electrical plug 6 which can be inserted in the electrical socket of the source 4 of electricity.

In other words, the management device 3 comprises an input for the electric current coming from the energy source and an output to which the electric apparatus 2 is connected.

In detail, the management device 3 comprises means for interrupting the current interposed between the input and the output in such a way as to interrupt the current during the interruption condition or allow the flow of current during the flow condition. It should be noted that the management device 3 may be implemented by any technically equivalent electrical device.

Moreover, the apparatus 1 comprises a measuring device 7 of at least a biometric value associated in use to the user's body for measuring the biometric value. More specifically, the measuring device 7 may be external such as, for example, a bracelet, a necklace, a belt, a chest strap, . . . or internal such as, for example, a chip or a detector under the skin.

In the preferred embodiment illustrated in the accompanying drawings, the measuring device 7 of the biometric value comprises a bracelet wearable on a user's arm and a sensor 14 for measuring the biometric value by contact with the user's body. Preferably, the sensor 14 for measuring the biometric value is a sensor for measuring a heartbeat. However, in other embodiments not expressly described here, the sensor 14 could measure other biometric values (adrenaline in the blood, . . . ).

More specifically, the device for measuring the biometric value may be a bracelet specially configured to measure a particular biometric value (FIGS. 2 and 3) or it may be integrated in a predetermined and already existing bracelet such as, for example, a watch. In detail, the bracelet may be a smartwatch 8 (preferably already configured for measuring the heartbeat) as illustrated in FIG. 1.

In accordance with this invention, the apparatus 1 comprises a processing unit 9 operatively connected to the management device 3 of the electric current and to the measuring device 7.

The processing unit 9 is configured for receiving a measuring signal 10 of the biometric value containing the biometric value measured by the measuring device 7. In other words, the measuring device 7 is configured to send the measuring signal 10 to the processing unit 9 where the measuring signal 10 contains the data relative to the measured biometric value.

Moreover, the processing unit 9 is configured for measuring a sudden variation in the measured biometric value relative to a reference biometric value. This biometric reference value may be calculated in an objective or subjective manner.

In any case, the reference biometric value indicates the presence of the user in a normal or customary or rest condition of life (from a medical point of view).

In other words, the processing unit 9 is configured to receive a signal containing the biometric reference value. The latter can be calculated by the apparatus 1 itself, or pre-memorised in an internal memory or the signal can arrive directly from the outside.

Preferably, the reference biometric value corresponds to one of the last measured biometric values or it is the average of the last measured biometric values. The term “last” is used to mean the biometric values measured most recently relative to the sudden variation of the reference biometric value.

In other words, the processing unit 9 is configured for comparing the measured biometric value with the reference biometric value. The latter may correspond to the most recent biometric values or may correspond to an independent predetermined biometric value.

Moreover, the processing unit 9 is configured for sending a disabling signal 11 to the management device 3 so as to configure the management device 3 from the enabled condition to the interruption condition when the biometric value distances from the reference biometric value by a reference differential value so as to block the motion of the part in motion of the apparatus 2. Obviously, the management device 3 is configured for receiving the disabling signal 11 and for configuring the latter from the enabling condition to the interruption condition.

More specifically, the processing unit 9 is configured for sending the disabling signal 11 to the management device 3 so as to configure the management device from the enabled condition to the interruption condition when the biometric value measured reaches or exceeds an altered biometric value representing a condition of presence of the user in a condition of altered life so as to disable the motion of the part of the apparatus 2. This altered biometric value is different from the reference biometric value by a differential reference value. Preferably, the altered biometric value is greater than the reference biometric value by the differential reference value.

In other words, in the case of sudden variation of the biometric value, the processing unit 9 is configured for disabling the movement of the part of the apparatus 2 to safeguard the user. The expression “sudden variation” means a variation of the biometric value over time which is faster than the variations, over time, the biometric value during the normal or customary life condition of the user.

In the preferred case, the measured biometric value is the heartbeat and the reference biometric value is defined by a heartbeat value in a condition of normal life of the user (e.g. number of heartbeats per minute), whilst the altered biometric value is defined by a heartbeat value greater than the heartbeat value of the normal life condition. In other words, when the value of heartbeats increases above a certain threshold, the processing unit 9 is configured to disable the movement of the part of the apparatus 2.

As already mentioned, if the movement is generated by movement means of the electric type the disabling signal 11 is used to disable the movement means or to interrupt the power supply signal. If the movement is generated by movement means of the combustion or other type, the disabling signal is used to act on the control system of the movement means.

In the preferred embodiment illustrated in the accompanying drawings, the processing unit 9 is configured for sending the disabling signal 11 to the management device 3 in order to configure the latter from the enabling condition to the interruption condition in such a way as to disconnect the apparatus 2 from the electric current. Obviously, the management device 3 is configured for receiving the disabling signal 11 and for configuring the latter from the enabling condition to the interruption condition.

In other words, in the case of sudden variation of the biometric value, the processing unit 9 is configured to disable the flow of electric current from the source 4 of electricity to the electric apparatus 2 in such a way as to interrupt the power supply of the latter so as to safeguard the user.

Advantageously, the invention enables the movement of the part in motion to be blocked when a sudden variation of the biometric value in question is detected (for example, an increase in the heart rate). In that way, it is possible to safeguard the user by interrupting the movement of the part of the electric apparatus 2 which the user is using.

It should be noted that the reference differential value is a function of the variation which the measured biometric must have to define a change such as to correspond to the occurrence of an event which is dangerous for the user.

In an embodiment of the invention illustrated in FIG. 3, the processing unit 9 is housed on the management device 3. In this way, the measuring device 7 (bracelet) measures the biometric value and transmits the measuring signal 10 (preferably via wireless) to the management device 3. In detail, the measuring signal 10 is received by the processing unit 9 which interrupts the movement (for example interrupting the supply of electric current). Even more in detail, the disabling signal 11 is transmitted inside the measuring device 7 between the processing unit 9 and the management device 3.

In an alternative embodiment, the processing unit 9 is housed on the measuring device 7 of the biometric value. In this way, the measuring signal 10 is transmitted inside the measuring device 7 towards the processing unit 9, whilst the disabling signal 11 is transmitted (preferably via wireless) by the processing unit 9 towards the management device 3. FIG. 3 illustrates a measuring device 7 integrated in a smartwatch 8 which is already configured to measure the biometric value (heartbeat) and is further configured for processing the measured biometric value as a function of a reference value and for sending the disabling signal 11 to the management device 3.

In a further embodiment illustrated in FIG. 2, the apparatus 1 comprises a smartphone 12 and/or a tablet PC and/or computer of known type comprising a relative processing unit. The processing unit 9 is defined by the processing card of the smartphone 12 and/or tablet and/or computer. In other words, the processing unit of the smartphone 12 and/or a tablet and/or computer is operatively connected to the measuring device 7 and to the management device 3 and is configured for:
receiving the measuring signal 10 of the biometric value containing the biometric value measured by the measuring device 7;
comparing the biometric value measured with the reference biometric value wherein the biometric reference value is represented by a condition of presence of the user in a condition of usual life;
sending the disabling signal 11 to the management device 3 so as to configure the management device from the enabled condition to the interruption condition when the biometric value measured reaches or exceeds the altered biometric value so as to disable the motion of the part of the apparatus 2.

It should be noted that the disabling signal 11 is transmitted from the smartphone 12 and/or tablet and/or computer to the management device 3 of the current via wireless. In the same way, the measuring signal 10 is transmitted by the measuring device 7 to the smartphone 12 and/or tablet and/or computer via wireless.

Lastly, it should be noted that the control unit is further configured for sending a re-enabling signal 13 to the management device 3 when the biometric value moves towards the reference biometric value relative to the differential value configuring the management device 3 from the interruption condition to the enabling condition.

This invention also relates to a method for safeguarding users during use of apparatuses 2 having at least a part in motion. The method is derived directly from what is described above with regard to the apparatus 1, which is here below incorporated in its entirety. More specifically, the method comprises a first step of connecting the management device 3 of the movement of the part of the apparatus 2 to the apparatus 2, wherein the management device 3 is configurable between the enabling condition and the interruption condition during which the movement of the part of the apparatus 2 is not is enabled.

Moreover, the method comprises a second step of measuring the biometric value of the user's body. Preferably, the second step is carried out by a sensor/detector 14 of the biometric value as described above.

In addition, the method comprises the step of comparing the measured biometric value with the reference biometric value in such a way as to check if there is a predetermined variation of the measured biometric value. In other words, it is detected if the measured biometric value moves away from the reference biometric value by the reference differential value in such a way as to disconnect the apparatus 2 from the electric current.

In that case, a disabling signal 11 is sent to the management device 3 so as to configure the management device 3 from the enabled condition to the interruption condition.

The invention achieves the preset aims.

More specifically, the invention makes it possible to safeguard users during the use of tools by interrupting the movement in the case of danger. In effect, in these dangerous situations the measuring device 7 detects a variation of a biometric parameter (e.g. heartbeat) of the user. The variation of the biometric parameter is the direct consequence of the user finding him/herself in a dangerous situation. Consequently, to avoid increasing the damage, the apparatus 1 according to this invention immediately switches OFF the current or disables the movement in another manner so as to disable the electrical apparatus 2.

In that way, the health of the users is safeguarded in a simple and optimum manner.

It should also be noted that this invention is relatively easy to implement and that the cost of implementing the invention is relatively low.

1. A management apparatus for safeguarding a user during use of a second apparatus that includes at least a part in motion, the management apparatus comprising:
a management device configured to manage a motion of the part of the second apparatus during use of the second apparatus, wherein said management device is configurable between an enabled condition during which the motion of the part of the second apparatus is enabled, and an interrupted condition during which the motion of the part of the second apparatus is disabled;
a measuring device configured to measure at least a biometric value associated with a body of the user while the management apparatus is in use, wherein the measuring device;
comprises a processing unit operatively connected to the management device of the motion and to the measuring device;
the processing unit configured to:
receive a measuring signal containing the measured biometric value measured by the measuring device;
compare the measured biometric value with a reference biometric value, wherein the biometric reference value represents a condition of a presence of the user in a normal condition;
send a disabling signal to the management device that changes the management device from the enabled condition to the interruption condition when the measured biometric value reaches or exceeds an altered biometric value representing a condition of the presence of the user in an abnormal condition so as to disable the motion of the part of the second apparatus, wherein the altered biometric value is different from the reference biometric value.

2. The management apparatus according to claim 1, wherein the processing unit is configured to disable the movement of the part of the second apparatus when the processing unit detects a sudden variation of the measured biometric value from the reference biometric value to the altered biometric value.

3. The management apparatus according to claim 1, wherein the altered biometric value is greater than the measured biometric value.

4. The management apparatus according to claim 1, wherein the management device, when in use, is connected to a control system of the part in motion in order to manage the part in motion.

5. The management apparatus according to claim 1, wherein the management device, when in use, is interposed between and connected to a source of electrical energy and the electrical supply of the second apparatus.

6. The management apparatus according to claim 1, wherein the reference biometric value is substantially equal to a most recently measured biometric value.

7. The management apparatus according to claim 1, wherein the reference biometric value is substantially equal to an average of a plurality of most recently measured biometric values.

8. The management apparatus according to claim 1, wherein the processing unit is housed on the management device.

9. The management apparatus according to claim 1, wherein the processing unit is housed on the measuring device of the biometric value.

10. The management apparatus according to claim 1, wherein the management apparatus comprises a smartphone and/or a tablet and/or a computer comprising its own processing card; the processing unit being defined by the processing card of the smartphone and/or tablet and/or computer.

11. The management apparatus according to claim 1, wherein the measuring device of the biometric value comprises a bracelet wearable on an arm of the user and a sensor for measuring the biometric value by contact with the user's body.

12. The management apparatus according to claim 1, wherein the measuring device for measuring the biometric value comprises a sensor for measuring a heartbeat in such a way that the heartbeat defines the biometric value, wherein the reference biometric value is defined by a heartbeat value in a condition of normal life of the user and the altered biometric value is defined by a heartbeat value greater than the heartbeat value of the normal life condition.

13. The management apparatus according to claim 1, wherein the disabling signal is transmitted by the processing unit to the management device via wireless communication.

14. The management apparatus according to claim 1, wherein the measuring signal is transmitted by the processing unit to the processing device via wireless communication.

15. A method for safeguarding a user during use of an apparatus that includes at least a part in motion, the method comprising:
connecting a management device configured to manage the part in motion of the apparatus to the apparatus, wherein the management device is configurable between an enabled condition during which the motion of the part of the apparatus is enabled, and an interruption condition during which the motion of the part of the apparatus is disabled;
measuring a biometric value of the user's body;
comparing the measured biometric value with a reference biometric value, wherein the reference biometric value is represented by a condition of a presence of the user in a normal condition;
sending a disabling signal to the management device to change the management device from the enabled condition to the interruption condition when the biometric value measured reaches or exceeds an altered biometric value that represents a condition of the presence of the user in an altered condition so as to disable the motion of the part of the apparatus, wherein the altered biometric value is different from the reference biometric value.
